(19) United States
(12) Patent Application Publication
(10) Pub. No.: US 2007/0179442 A1
(43) Pub. Date: Aug. 2, 2007

(54) Single-use syringe

(76) Inventors: Ming-Jeng Shue, Taichung City (TW); Phillip Shue, Taichung City (TW); Deborah Huang, Taichung City (TW)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/520,339

(22) Filed: Sep. 12, 2006

(30) Foreign Application Priority Data
Jan. 27, 2006 (TW) 095103442

(51) Int. Cl. A61M 5/00

(52) U.S. Cl. 604/110

(57) ABSTRACT

A single-use syringe includes a deformable sealing member which is in a fluid-tight and slidable engagement with a barrel wall, a plunger which is disposed to be movable along the barrel wall, a first coupling member which is secured to a front end wall of the plunger, and which has a first connected end disposed forwardly of the front end wall, and a second coupling member which is secured to a trail end wall of the sealing member, and which has a second connected end disposed rearwardly of the trail end wall. The second connected end confronts the first connected end in a radial direction, and is attached to the first connected end at a bonding region. Immediately after the second connected end stays put as a result of abutment of the sealing member against a shoulder of the barrel, a subsequent forward force applied to the plunger breaks the bonding region, thereby preventing operation of the plunger to move the sealing member rearward, and thereby rendering the syringe unreusable.

### CROSS-REFERENCE TO RELATED APPLICATION

This application claims priority of Taiwanese patent Application No. 095103442, filed on Jan. 27, 2006.

### BACKGROUND OF THE INVENTION

1. Field of the Invention

This invention relates to a single-use syringe, more particularly to a single-use syringe, in which a deformable sealing member is separated from a plunger after use so as to render the syringe unreusable.

2. Description of the Related Art

Referring to FIGS. 1 and 2, a conventional single-use syringe is shown to include a barrel 91 which has an axially extending surrounding wall 913 defining front and rear passageways 912,911, and a surrounding shoulder 914 disposed between the front and rear passageways 912,911, a needle assembly 92 which is sleeved on a front opened end of the surrounding wall 913, a plunger 93 which is disposed to be movable in and along the rear passageway 911 and which has a tapered head 931 formed on a front end thereof, a deformable sealing member 94 which is retainingly sleeved on the tapered head 931, which is in a fluid-tight and slidable engagement with the surrounding wall 913 at the rear passageway 911, and which has an axially extending vent hole 932 communicated with the ambient air, and a plug 95 which is detachably connected to the deformable sealing member 94 and which closes the vent hole 932. An inner surface of the surrounding wall 913 has a retaining ring 915 formed adjacent to the shoulder 914. The plug 95 has an anchoring portion 951 projecting forwardly and outwardly of the deformable sealing member 94 such that, subsequent to completion of injection, the deformable sealing member 94 is brought to abut against the shoulder 914, and the anchoring portion 951 is engaged with the retaining ring 915 so as to permit disengagement of the plug 95 from the deformable sealing member 94 when the plunger 93 is pulled rearwardly, and so as to disrupt the fluid-tightness of the vent hole 932 to thereby render the syringe unreusable. However, when the anchoring portion 951 is forced to be engaged with the retaining ring 915, the frictional force between the plug 95 and the deformable sealing member 94 is increased, which may be greater than the force of engagement between the anchoring portion 951 and the retaining ring 915, thereby adversely affecting separation of the plug 95 from the deformable sealing member 94. On the other hand, medication fluid may flow into the vent hole 932 due to insufficient tightness between the plug 95 and the deformable sealing member 94. Moreover, it is desirable to provide a shield for the used needle tip of the needle assembly 92 for safe disposal.

In U.S. Pat. No. 5,000,735, a single use syringe is disclosed, which has a plunger that is separated from a piston inside of a barrel after a downward stroke of the plunger so as to prevent reuse of the syringe. The plunger breaks breakaway portions mounted inside the piston when pushed downward to unload the syringe so as to separate the plunger from the piston and destroy the syringe, thereby preventing the piston from being withdrawn for the purpose of reloading the syringe. Since the breakaway portions are disposed inside the piston, the syringe is complicated in structure and is inconvenient to manufacture and assemble.

### SUMMARY OF THE INVENTION

An object of the present invention is to provide a single-use syringe which can prevent reuse, and which has a simple construction to facilitate manufacture and assembly.

Another object of the present invention is to provide a single-use syringe which can minimize the amount of residual injection fluid.

Still another object of the present invention is to provide a single-use syringe which has an encasing member that shields a used needle tip for safe disposal.

According to this invention, the single-use syringe includes a barrel having front and rear barrel ends opposite to each other along an axis in an axial direction, and a surrounding barrel wall which interconnects the front and rear barrel ends, and which includes small-diameter and large-diameter wall portions that are disposed proximate to the front and rear barrel ends, respectively, and that respectively have front and rear inner wall surfaces respectively defining a passage and an accommodation chamber which are in fluid communication with each other, and a shoulder that is interposed between the small-diameter and large-diameter wall portions. A needle cannula has a rear secured end and a front tip end opposite to each other along the axis. The rear secured end is affixed to the small-diameter wall portion to be communicated with the passage such that the front tip end is exposed outwardly of the front barrel end in use. A plunger is disposed to be movable in the accommodation chamber along the large-diameter wall portion. The plunger has a front end wall which confronts the passage, and a rear end wall which extends outwardly of the rear barrel end to be manually operable. A hollow deformable sealing member includes ahead end wall which confronts the shoulder, and a deformable surrounding wall which extends from the head end wall rearwardly, which is in a fluid-tight and slidable engagement with the large-diameter wall portion so as to permit the head end wall to be brought to abut against the shoulder, and which terminates at a trail end wall. A first coupling member includes a first secured end which is secured to the front end wall, and a first connected end which is disposed opposite to the first secured end in the axial direction and forwardly of the front end wall. A second coupling member includes a second secured end which is secured to the trail end wall, and a second connected end which is opposite to the second secured end and rearwardly of the trail end wall. The second connected end confronts the first connected end in a radial direction relative to the axis, and is attached to the first connected end at a bonding region with a bonding force such that the bonding force is greater than a frictional force generated between the deformable surrounding wall and the large-diameter wall portion when the plunger is forced to move in the accommodation chamber, and such that immediately after the second connected end stays put as a result of abutment of the head end wall against the shoulder, a subsequent forward force applied to the rear end wall of the plunger imparts to the bonding region a shear force which breaks the bonding region when the shear force is greater than the bonding force, thereby preventing operation of the plunger to move the deformable sealing member rearward, and thereby rendering the syringe unreusable.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 is a sectional view of a conventional single-use syringe;

FIG. 2 is a sectional view of the conventional single-use syringe of FIG. 1 after use;

FIG. 3 is an exploded sectional view of the first preferred embodiment of a single-use syringe according to this invention;

FIG. 4 is a sectional view of the first preferred embodiment during an injection stroke;

FIG. 5 is a sectional view showing the first preferred embodiment in a state after completion of the injection stroke;

FIGS. 6 to 8 are fragmentary sectional views of the first preferred embodiment, showing how a deformable sealing member is separated from a plunger of the syringe;

FIG. 9 is a sectional view of the second preferred embodiment of a single-use syringe according to this invention;

FIG. 10 is a sectional view showing the second preferred embodiment in a state where a deformable sealing member is separated from a plunger of the syringe;

FIG. 11 is a sectional view of the third preferred embodiment of a single-use syringe according to this invention;

FIG. 12 is a fragmentary sectional view showing the third preferred embodiment in a state after completion of the injection stroke;

FIG. 13 is a sectional view showing the third preferred embodiment in a state where a deformable sealing member is separated from a plunger of the syringe;

FIGS. 14 and 15 are fragmentary sectional views of the fourth preferred embodiment of a single-use syringe according to this invention;

FIGS. 16 and 17 are fragmentary sectional views of the fifth preferred embodiment of a single-use syringe according to this invention;

FIG. 18 is a fragmentary sectional view of the sixth preferred embodiment of a single-use syringe according to this invention;

FIGS. 19 and 20 are fragmentary sectional views of the seventh preferred embodiment of a single-use syringe according to this invention;

FIG. 21 is a fragmentary sectional view of the eighth preferred embodiment of a single-use syringe according to this invention;

FIG. 22 is a sectional view of the ninth preferred embodiment of a single-use syringe according to this invention;

FIGS. 23 to 25 are sectional views of the tenth preferred embodiment of a single-use syringe according to this invention, showing how a deformable sealing member is separated from a plunger of the syringe;

FIG. 26 is a sectional view of the eleventh preferred embodiment of a single-use syringe according to this invention;

FIG. 27 is a sectional view showing the eleventh preferred embodiment in a state after completion of the injection stroke; and

FIG. 28 is a sectional view showing the eleventh preferred embodiment in a state where a deformable sealing member is separated from a plunger of the syringe.

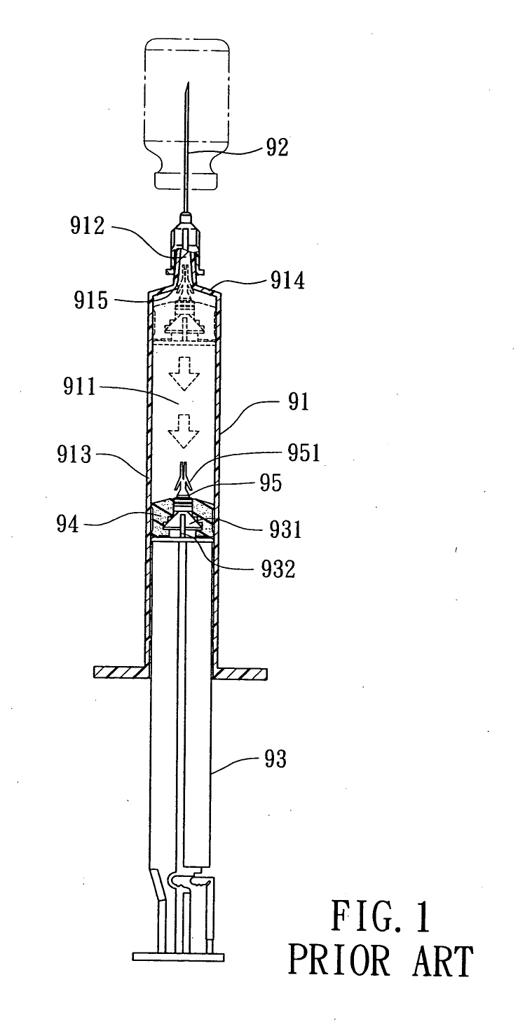

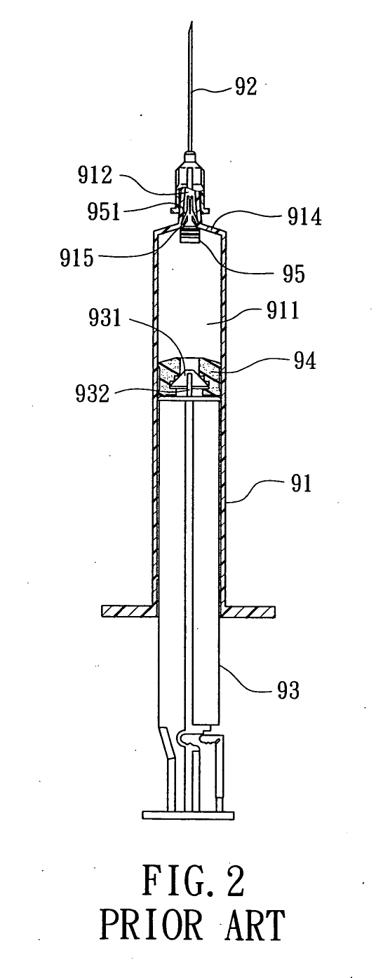

### DETAILED DESCRIPTION OF THE PREFERRED EMBODIMENTS

Before the present invention is described in greater detail, it should be noted that same reference numerals have been used to denote like elements throughout the specification.

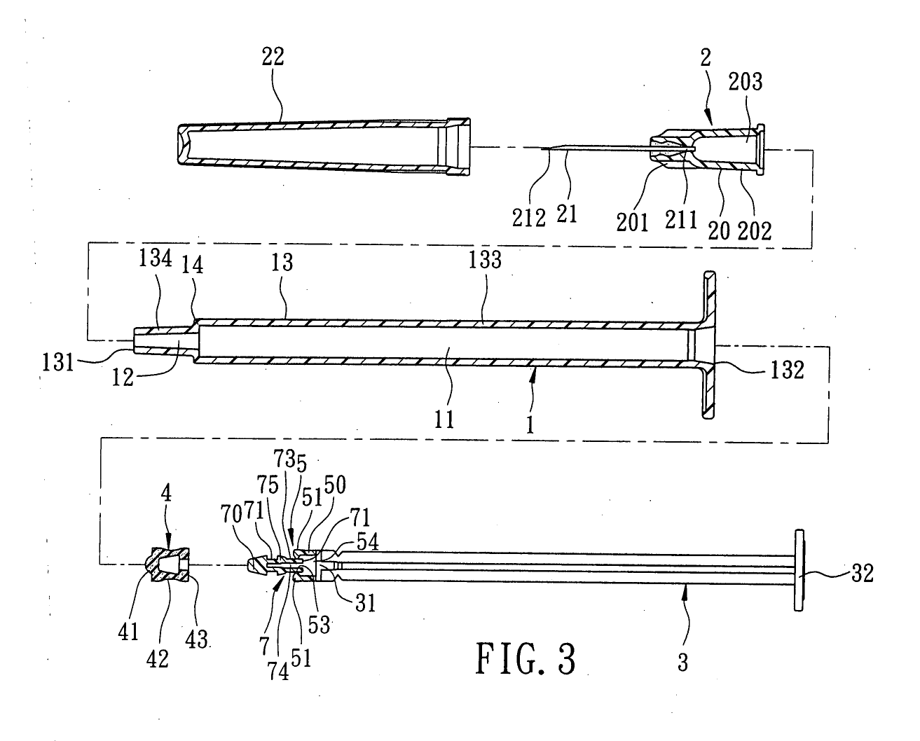

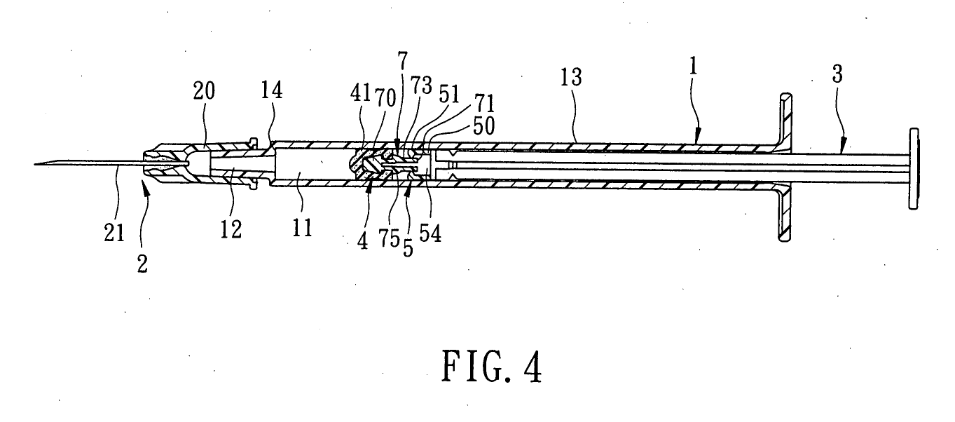

Referring to FIGS. 3 and 4, the first preferred embodiment of a single-use syringe according to the present invention is shown to comprise a barrel 1, a needle assembly 2, a plunger 3, a hollow deformable sealing member 4, and first and second coupling members 5,7.

In this embodiment, the barrel 1 is used for a small-volume injection, such as 1 ml, and includes front and rear barrel ends 131,132 opposite to each other along an axis in an axial direction, and a surrounding barrel wall 13 which interconnects the front and rear barrel ends 131,132, and which includes small-diameter and large-diameter wall portions 134,133 that are disposed proximate to the front and rear barrel ends 131,132, respectively, and that respectively have front and rear inner wall surfaces defining a passage 12 and an accommodation chamber 11, respectively, which are in fluid communication with each other, and a shoulder 14 that is interposed between the small-diameter and large-diameter wall portions 134,133.

The needle assembly 2 includes a tubular needle seat 20, a needle cannula 21 and a tip protector 22. The needle seat 20 has a tubular retaining portion 201 which retains a rear secured end 211 of the needle cannula 21 to permit a front tip end 212 that is opposite to the rear secured end 211 along the axis to extend forward, from the tubular retaining portion 201, and a tubular sleeve portion 202 which is opposite to the tubular retaining portion 201 along the axis to define a duct 203 that is fluidly communicated with the needle cannula 21, and which is detachably sleeved on the small-diameter wall portion 134 to fluidly communicate the duct 203 with the passage 12. The tip protector 22 is removable and is in a fluid-tight engagement with the tubular sleeve portion 202 for shielding the needle cannula 21 before use.

The plunger 3 is disposed to be movable in the accommodation chamber 11 along the large-diameter wall portion 13, and has a front end wall 31 which confronts the passage 12, and a rear end wall 32 which extends outwardly of the rear barrel end 132 to be manually operable.

The deformable sealing member 4 has a head end wall 41 which confronts the shoulder 14, and a deformable surrounding wall 42 which extends from the head end wall 41 rearwardly, which is in a fluid-tight and slidable engagement with the large-diameter wall portion 133 so as to permit the head end wall 41 to be brought to abut against the shoulder 14, and which terminates at a trail end wall 43.

The first coupling member 5 has a first secured end 50 which is secured to the front end wall 31, and left and right sliding units 51 which extend forwardly from the first secured end 50 in the axial direction and which cooperatively serve as a first connected end. The left and right sliding units 51 are spaced apart from each other in a radial direction relative to the axis to confine an axial channel 54 therebetween.

The second coupling member 7 has a second secured end 70 which is secured to the deformable sealing member 4, and left and right bendable units 71 which extend rearwardly from the second secured end 70 in the axial direction and which are disposed rearwardly of the trail end wall 43 to cooperatively serve as a second connected end. The left and right bendable units 71 are spaced apart from each other in the radial direction to define an axial clearance 74, and respectively confront the left and right sliding units 51 in the radial direction. The left and right bendable units 71 are attached to the left and right sliding units 51, respectively, at bonding regions 53 with a bonding force. The bonding force is greater than a frictional force generated between the deformable surrounding wall 42 and the large-diameter wall portion 133 when the plunger 3 is forced to move in the accommodation chamber 11. Each of the left and right bendable units 71 has an outer sliding surface 73 which extends in the axial direction. Preferably, the outer sliding surface 73 has an inclined segment 75 which is disposed proximate to the second secured end 70 and which is inclined from the axis to the second secured end 70.

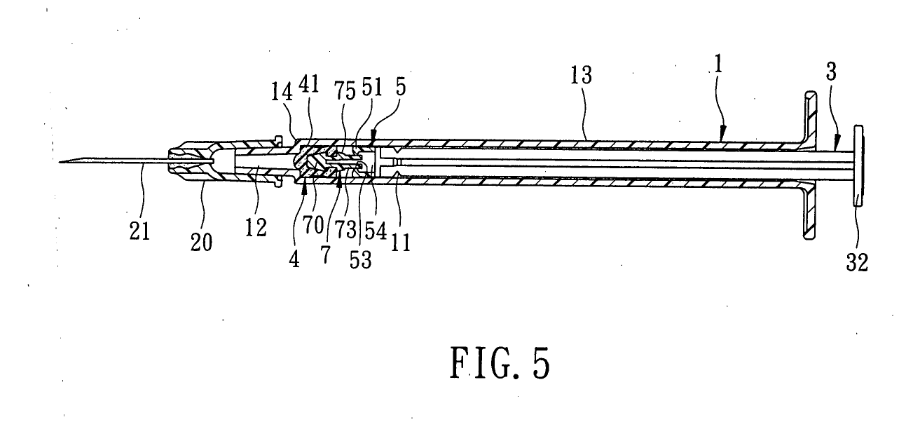

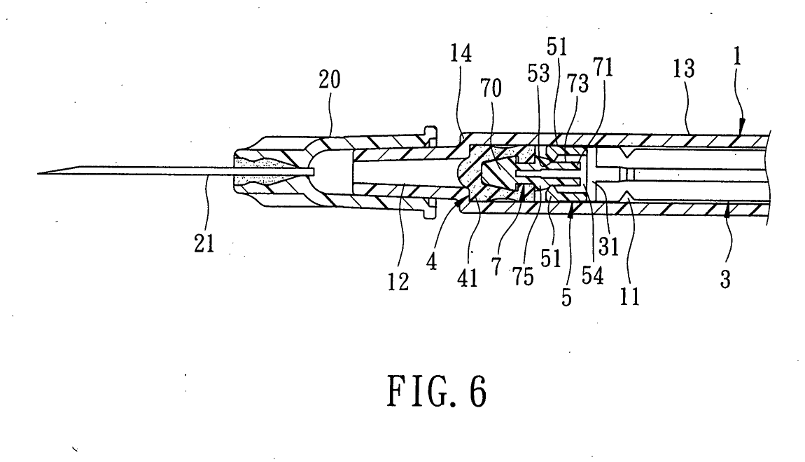

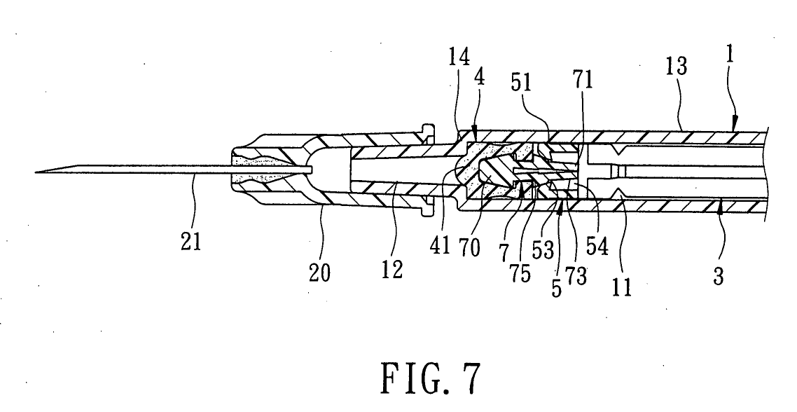

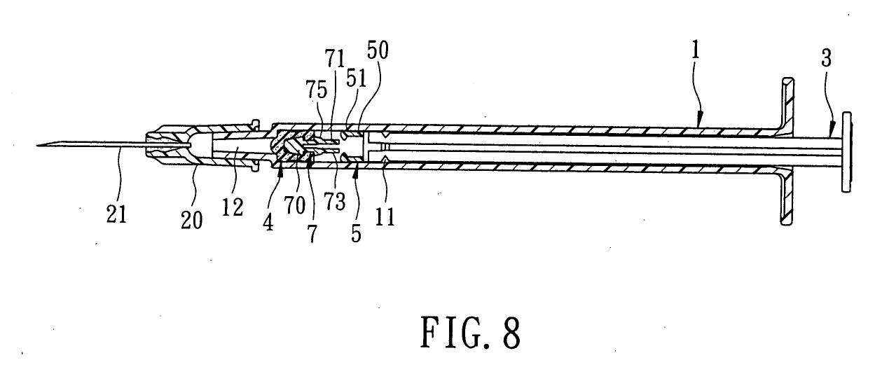

As shown in FIGS. 5 and 6, after an injection procedure is completed, and immediately after the second connected end 70 stays put as a result of abutment of the head end wall 41 against the shoulder 14, a subsequent forward force applied to the rear end wall 32 of the plunger 3 imparts to the bonding regions 53 a shear force which breaks the bonding regions 53 when the shear force is greater than the bonding force such that the bonding regions 53 are forced to slide on the outer sliding surfaces 73 and such that the left and right bendable units 71 are accommodated in the axial channel 54. Subsequently, as shown in FIG. 7, when the bonding regions 53 slide along the inclined segments 75, with the axial clearance 74 providing a leeway, the left and right bendable units 71 are bent toward the axis so as to acquire a biasing force that imparts to the bonding regions 53, an increased force component in the axial direction to thereby facilitate rearward movement of the bonding regions 53 relative to the inclined segments 75, as shown in FIG. 8. Thus, operation of the plunger 3 to move the deformable sealing member 4 rearward is prevented to thereby render the syringe unreusable.

It should be appreciated that the left and right sliding units 51 and the left and right bendable units 71 are interchangeable.

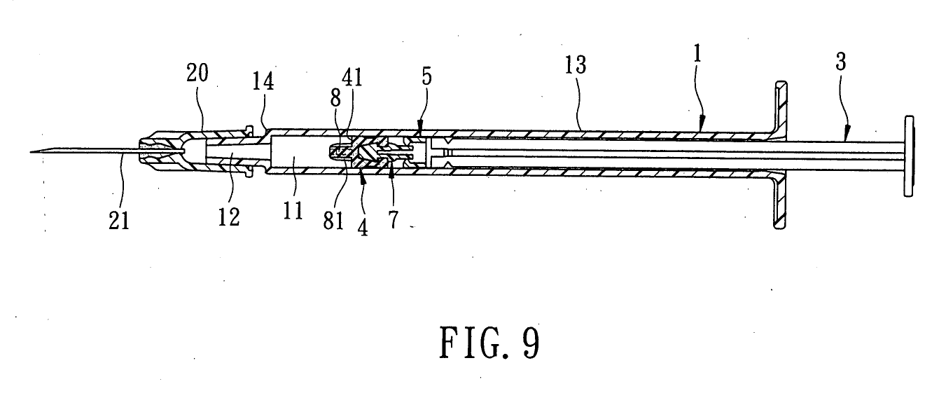

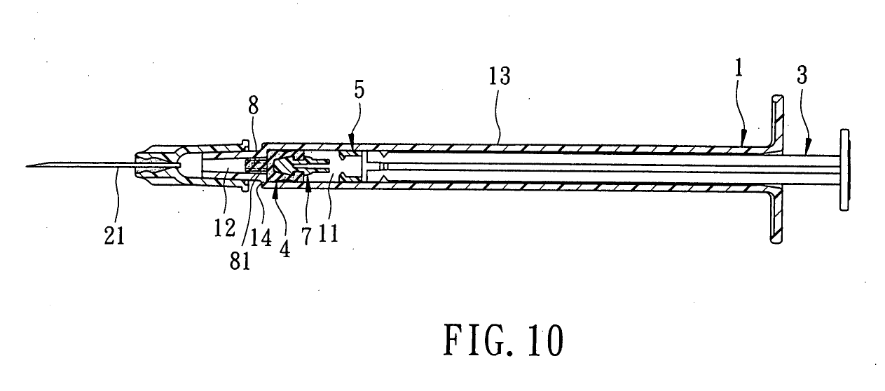

Referring to FIGS. 9 and 10, the second preferred embodiment of a single-use syringe according to this invention is shown to be similar to the first embodiment in construction. The syringe of this embodiment further comprises a plug 8 which is integrally formed with and which extends forwardly from the head end wall 41 of the deformable sealing member 4 such that when the head end wall 41 is brought to abut against the shoulder 14, the plug 8 is forced to plug the passage 12. The plug 8 has frictional ribs 81 configured to frictionally engage an inner surface of the small-diameter wall portion 134 with enhanced friction. Moreover, a gap is formed between each adjacent pair of the frictional ribs 81 so as to facilitate flowing of injection fluid into the passage 12.

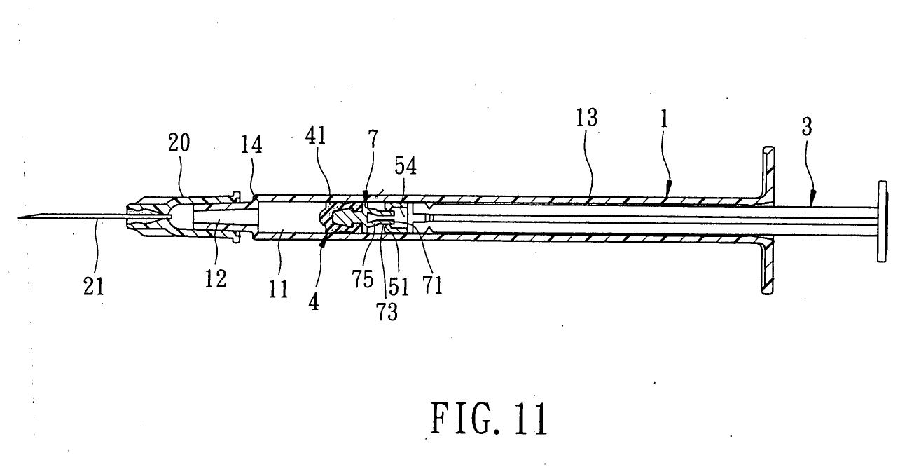

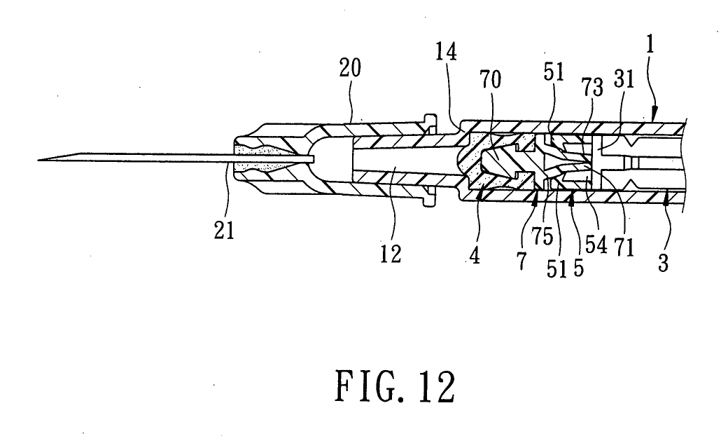

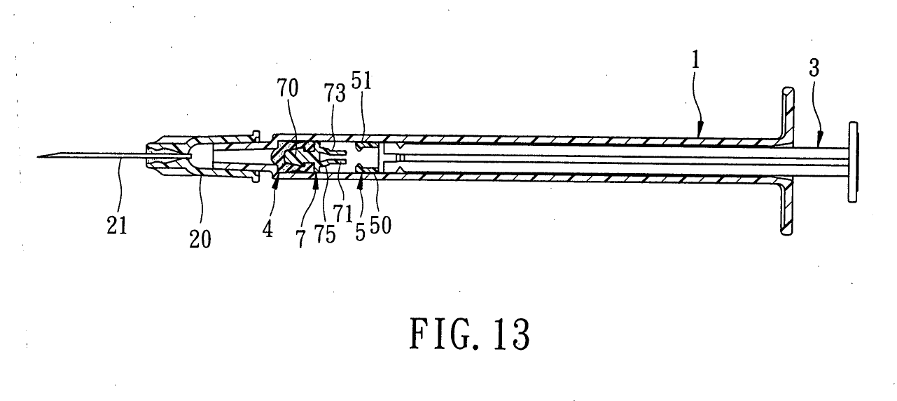

Referring to FIGS. 11 to 13, the third preferred embodiment of a single-use syringe according to this invention is shown to be similar to the first embodiment in construction. The second coupling member 7 of this embodiment slightly differs from that of the first embodiment in configuration.

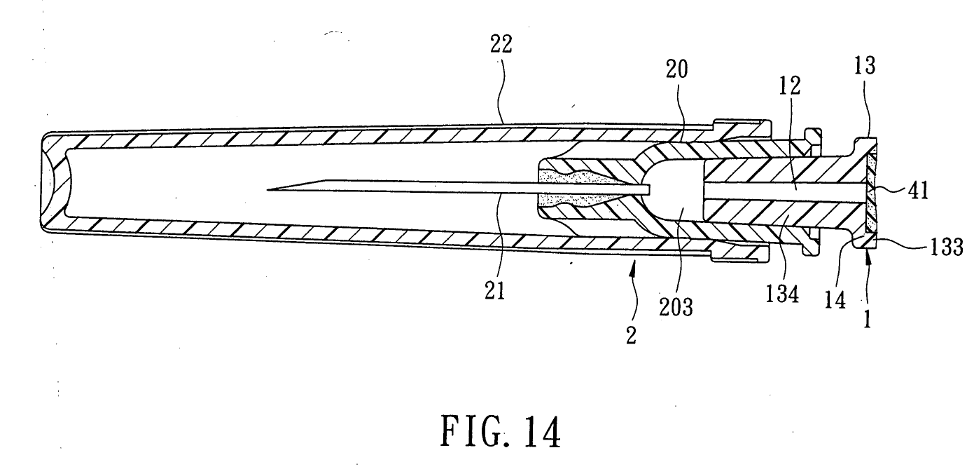

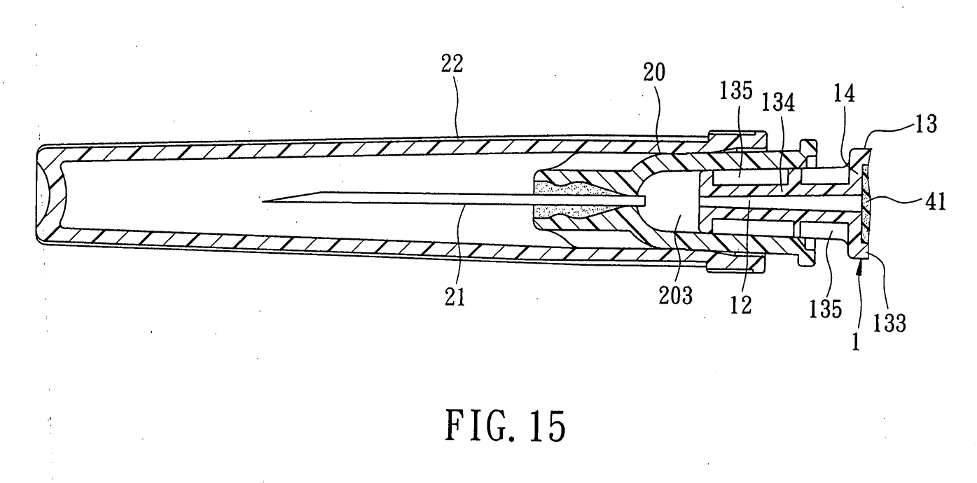

Referring to FIG. 14, the fourth preferred embodiment of a single-use syringe according to this invention is shown to be similar to the first embodiment in construction. The difference resides in that the surrounding barrel wall 13 has a larger thickness at the small-diameter wall portion 134, and a smaller thickness at the large-diameter wall portion 133. Thus, the amount of residual injection fluid in the barrel 1 is minimized. In a modified form of the fourth preferred embodiment of a single-use syringe according to this invention as shown in FIG. 15, the small-diameter wall portion 134 has a plurality of angularly-displaced ribs 135 formed on an outer surface thereof.

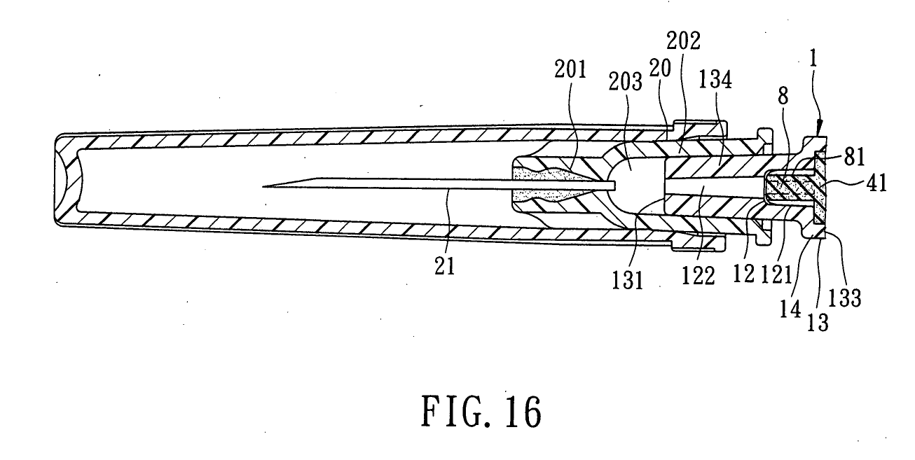

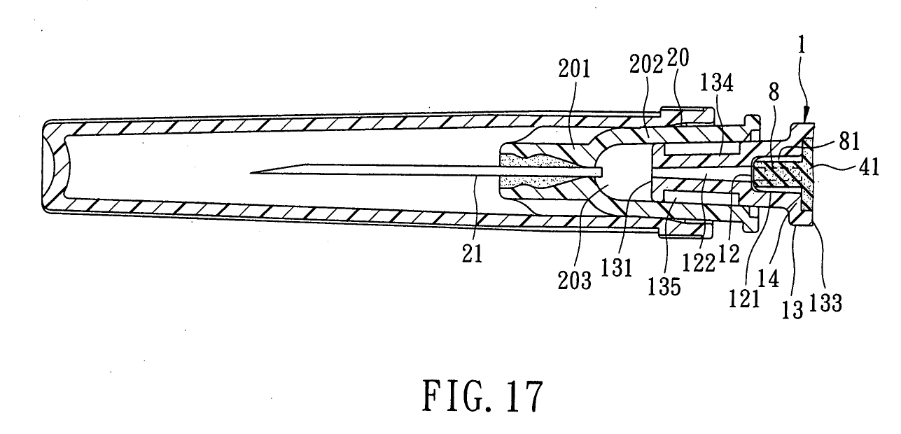

Referring to FIG. 16, the fifth preferred embodiment of a single-use syringe according to this invention is shown to be similar to the second embodiment in construction. The difference resides in that the passage 12 includes a wider passage segment 121 which extends from the shoulder 14 forwardly and which is dimensioned such that the plug 8 is tightly fitted thereinto, and a narrower passage segment 122 which is disposed between the wider passage segment 121 and the front barrel end 131. Thus, the amount of residual injection fluid in the barrel 1 is minimized. In a modified form of the fifth preferred embodiment of a single-use syringe according to this invention as shown in FIG. 17, the small-diameter wall portion 134 has a plurality of angularly-displaced ribs 135 formed on an outer surface thereof.

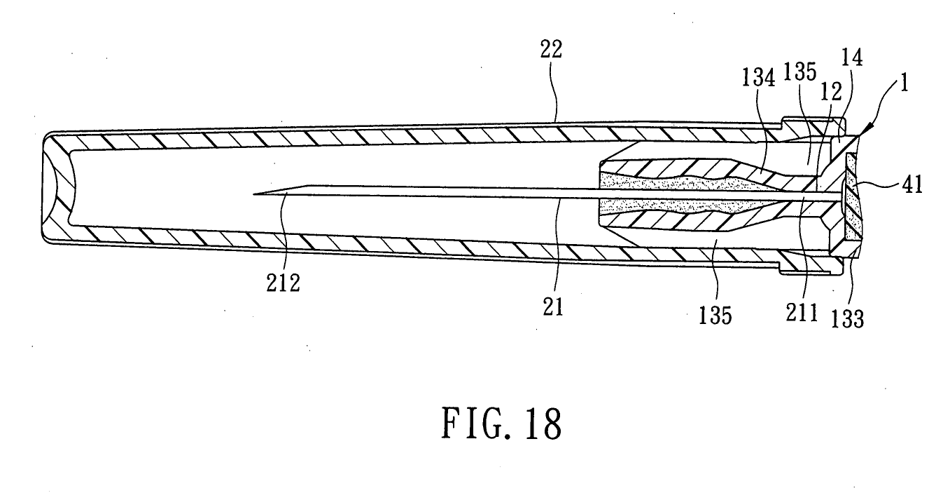

Referring to FIG. 18, the sixth preferred embodiment of a single-use syringe according to this invention is shown to have a construction similar to that of the fourth embodiment as shown in FIG. 15. The difference resides in that the small-diameter wall portion 134 has an inner grip surface which defines the passage 12 and which is configured such that the rear secured end 211 of the needle cannula 21 is adhered to and is in a fluid-tight engagement with the inner grip surface. Thus, the needle seat 20 is dispensed with. Further, the tip protector 22 is disposed to be in a fluid-tight engagement with a portion of the large-diameter wall portion 133 adjacent to the shoulder 14.

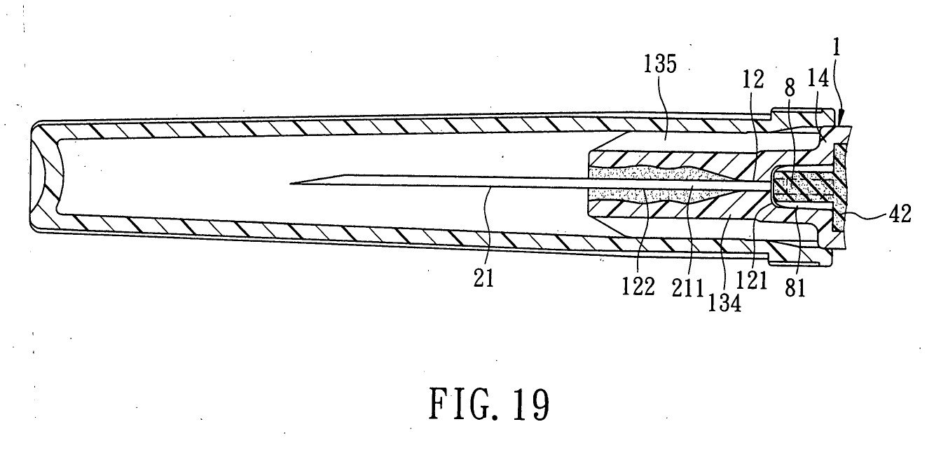

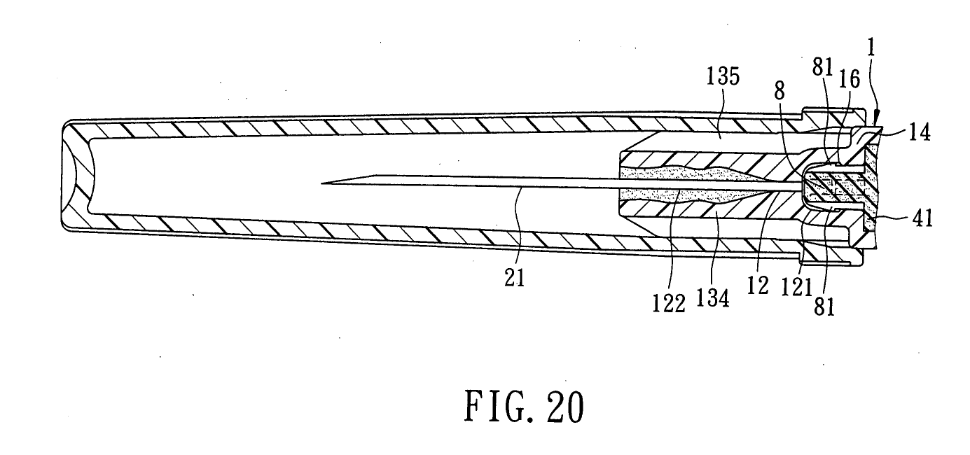

Referring to FIG. 19, the seventh preferred embodiment of a single-use syringe according to this invention is shown to be similar to the sixth embodiment in construction. In the seventh embodiment, the small-diameter wall portion 134 of the surrounding barrel wall 13 has an inner grip surface which defines a narrower passage segment 122 and which is configured such that the rear secured end 211 of the needle cannula 21 is adhered to and is in a fluid-tight engagement with the inner grip surface, and a wider passage segment 121 which is dimensioned such that a plug 8 is tightly fitted thereinto. Further, as shown in FIG. 20, the small-diameter wall portion 134 further has an annular step 16 formed in the wider passage segment 121 so as to tightly engage the frictional ribs 81 of the plug 8 for enhancing the frictional engagement between the small-diameter wall portion 134 and the plug 8.

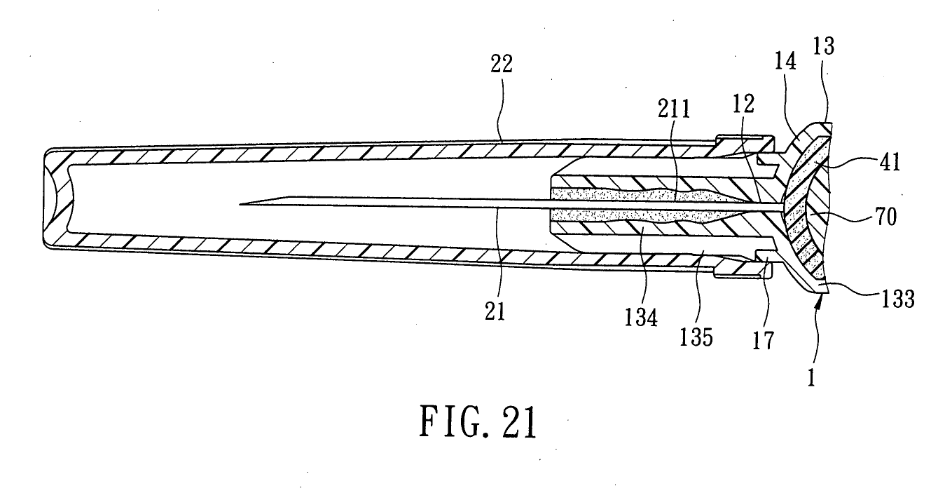

Referring to FIG. 21, the eighth preferred embodiment of a single-use syringe according to this invention is shown to be similar to the sixth embodiment in construction. In the eighth embodiment, an annular rib 17 is formed on the small-diameter wall portion 134 of the surrounding barrel wall 13 adjacent to the shoulder 14 such that the tip protector 22 is in air-tight contact with the annular rib 17, thereby preventing entry of dust into the tip protector 22.

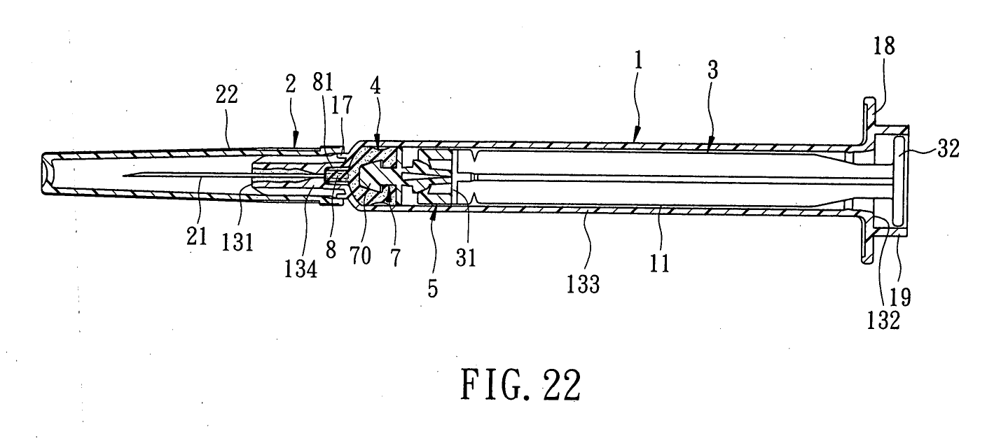

Referring to FIG. 22, the ninth preferred embodiment of a single-use syringe according to this invention is shown to be similar to the second embodiment in construction. In the ninth embodiment, the barrel 1 further includes a tubular guard portion 19 which extends rearwardly from the rear barrel end 132 and which defines a receiving space communicated with the accommodation chamber 11. The plunger 3 has a rear end wall 32 in the form of a thumb rest which extends rearwardly and outwardly of the tubular guard portion 19 for manual operation, and which is received in the receiving space when the plunger 3 reaches the shoulder 14 so that the plunger 3 is difficult to be pulled out again for reuse.

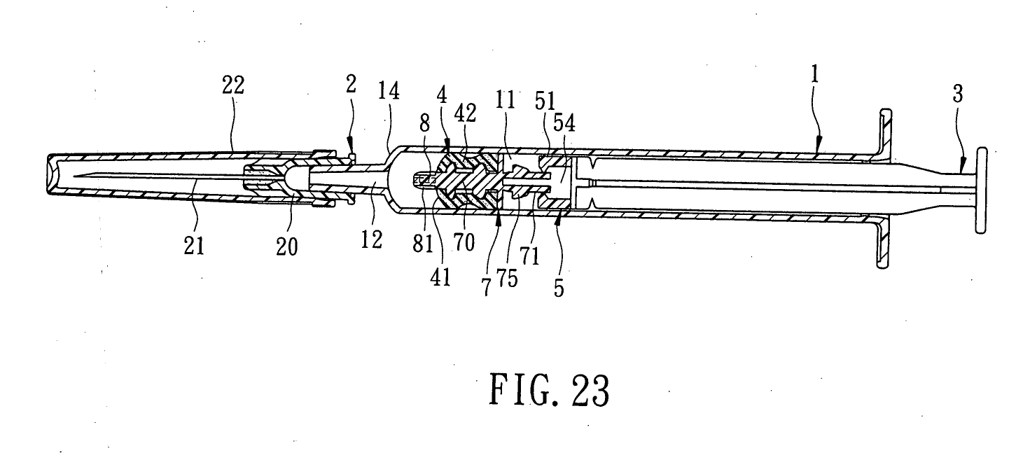

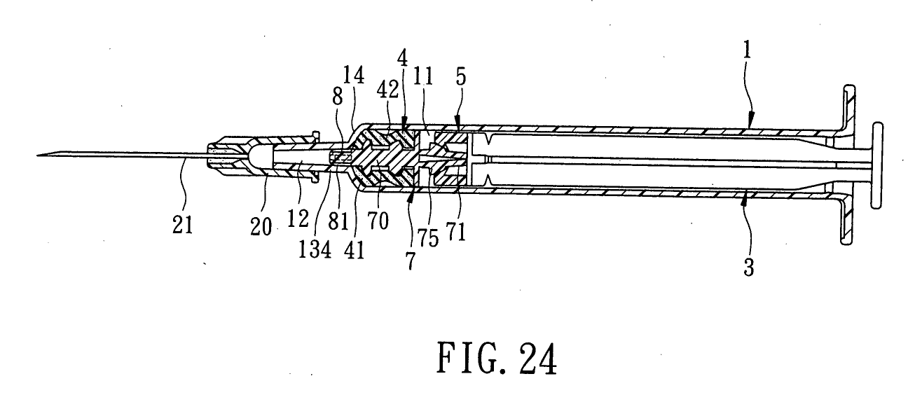

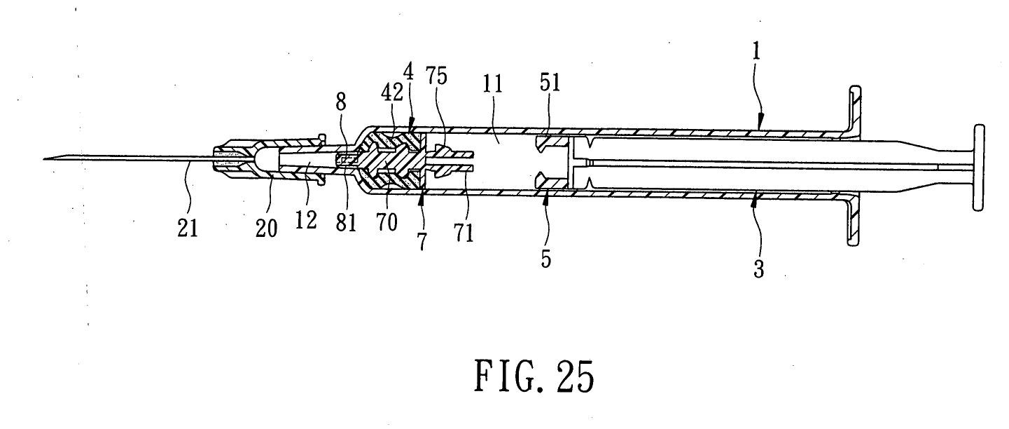

Referring to FIGS. 23 to 25, the tenth preferred embodiment of a single-use syringe according to this invention is shown to be similar to the previous embodiments in construction, and is used for large-volume injection, such as 3 ml or 5 ml. In this embodiment, the second coupling member 7 slightly differs from that of the previous embodiments in configuration. A plug 8 is integrally formed with the second connected end 70 of the second coupling member 7, and extends forwardly of the head end wall 41 of the deformable sealing member 4 such that when the head end wall 41 is brought to abut against the shoulder 14, the plug 8 is forced to plug the passage 12. The plug 8 has frictional ribs 81 configured to frictionally engage an inner surface of the small-diameter wall portion 134 with enhanced friction, and defining a plurality of gaps to facilitate flowing of injection fluid into the passage 12.

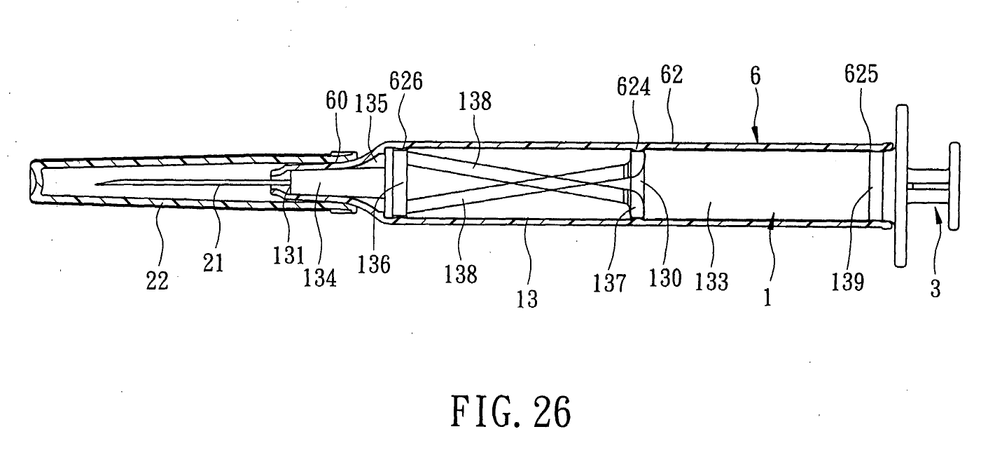

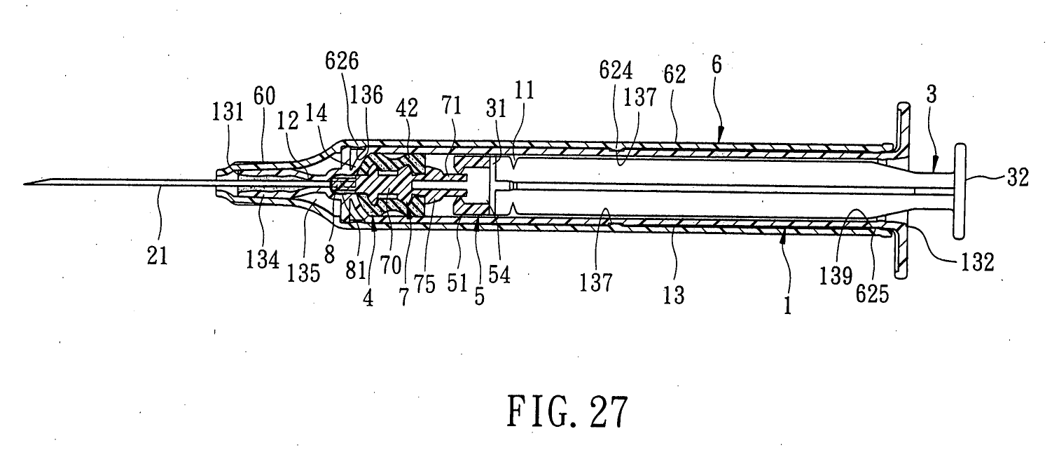

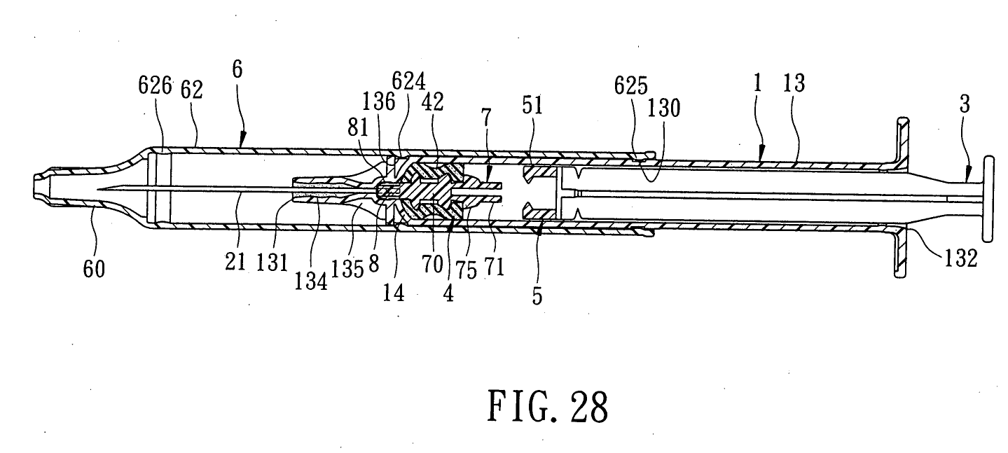

Referring to FIGS. 26 to 28, the eleventh preferred embodiment of a single-use syringe according to this invention is shown to be similar to the previous embodiments in construction. In this embodiment, an encasing member 6 is configured to be sleevable on the surrounding barrel wall 13 from the front barrel end 131. The encasing member 6 includes surrounding front and rear segments 60,62. The surrounding front segment 60 surrounds the small-diameter wall portion 134, and permits the needle cannula 21 to extend outwardly thereof and to be retractable thereinto. The surrounding rear segment 62 extends from the surrounding front segment 60 in the longitudinal direction to be sleeved on the large-diameter wall portion 133, and is movable relative to the barrel 1 between a position of use, as shown in FIG. 27, where the small-diameter wall portion 134 is closer to the surrounding front segment 60, and where the needle cannula 21 extends outwardly of the surrounding front segment 60, and a disposal position, as shown in FIG. 28, where the small-diameter wall portion 134 is remote from the surrounding front segment 60, and where the needle cannula 21 is disposed inwardly of the surrounding front segment 60 as a result of withdrawal of the small-diameter wall portion 134 from the surrounding front segment 60. The large-diameter wall portion 133 and the rear surrounding segment 62 respectively have a plurality of spirally extending recesses 137 and a plurality of protrusions 624 which are angularly engageable when the encasing member 6 is sleeved on the barrel 1 so as to prevent the surrounding barrel wall 13 from displacing along the surrounding rear segment 62 in the axial direction, thereby placing the surrounding barrel wall 13 in the position of use. The recesses 137 and the protrusions 624 are configured such that the surrounding barrel wall 13 is angularly movable relative to the surrounding rear segment 62 so as to enable subsequent axial displacement of the surrounding barrel wall 13 from the position of use to the disposal position. In addition, the large-diameter wall portion 133 of the surrounding barrel wall 13 further has a surrounding retaining groove 136 which is formed adjacent to the shoulder 14 and which is opposite to the spirally extending recesses 137 in the axial direction so as to engage the protrusions 624 when the surrounding barrel wall 13 is in the disposal position, as shown in FIG. 28, thereby preventing the surrounding barrel wall 13 from displacing along the surrounding rear segment 62. Moreover, the large-diameter wall portion 133 of the surrounding barrel wall 13 further has a plurality of guideways 138 which extend to interconnect the spirally extending recesses 137 and the surrounding retaining groove 136 so as to facilitate the displacement of the surrounding barrel wall 13 from the position of use to the disposal position.

In addition, the surrounding rear segment 62 has first and second retaining rings 626,625. The large-diameter wall portion 133 has first and second retaining grooves 130,139. Thus, when the surrounding barrel wall 13 is in the position of use, the first and second retaining rings 626,625 are engaged with the retaining grooves 136,139, respectively (see FIG. 26). When the surrounding barrel wall 13 is in the disposal position, the second retaining ring 625 is engaged with the first retaining groove 130 (see FIG. 28).

Furthermore, a tip protector 22 is detachably sleeved on the surrounding front segment 60 of the encasing member 6 for shielding the needle cannula 21 before use.

As illustrated, in the single-use syringe of this invention, by virtue of the abutment of the head end wall 41 against the shoulder 14, a subsequent forward force applied to the plunger 3 by the user can break the bonding region 53, thereby preventing operation of the plunger 3 to move the deformable sealing member 4 rearward. Thus, the syringe can be conveniently rendered unreusable after injection in a single operation. In addition, since the first and second connected ends 51,71 which interconnect the deformable sealing member 4 and the plunger 3 are disposed outwardly of the deformable sealing member 4, the air-tight engagement between the deformable sealing member 4 and the barrel wall 13 can be maintained, and the syringe is convenient to assemble and manufacture. Besides, in one of the embodiments, the used needle cannula 21 can be withdrawn into the encasing member 6 for safe disposal.

While the present invention has been described in connection with what are considered the most practical and preferred embodiments, it is understood that this invention is not limited to the disclosed embodiments but is intended to cover various arrangements included within the spirit and scope of the broadest interpretations and equivalent arrangements.

We claim:

1. A single-use syringe comprising:
a barrel including front and rear barrel ends opposite to each other along an axis in an axial direction, and a surrounding barrel wall which interconnects said front and rear barrel ends, and which includes
small-diameter and large-diameter wall portions that are disposed proximate to said front and rear barrel ends, respectively, and that respectively have front and rear inner wall surfaces respectively defining a passage and an accommodation chamber which are in fluid communication with each other, and
a shoulder that is interposed between said small-diameter and large-diameter wall portions;
a needle cannula having a rear secured end and a front tip end opposite to each other along the axis, said rear secured end being affixed to said small-diameter wall portion to be communicated with said passage such that said front tip end is exposed outwardly of said front barrel end in use;
a plunger which is disposed to be movable in said accommodation chamber along said large-diameter wall portion, said plunger having a front end wall which confronts said passage, and a rear end wall which extends outwardly of said rear barrel end to be manually operable;
a hollow deformable sealing member including a head end wall which confronts said shoulder, and a deformable surrounding wall which extends from said head end wall rearwardly, which is in a fluid-tight and slidable engagement with said large-diameter wall portion so as to permit said head end wall to be brought to abut against said shoulder, and which terminates at a trail end wall;
a first coupling member including a first secured end which is secured to said front end wall, and a first connected end which is disposed opposite to said first secured end in the axial direction and forwardly of said front end wall; and
a second coupling member including a second secured end which is secured to said trail end wall, and a second connected end which is disposed opposite to said second secured end and rearwardly of said trail end wall, said second connected end confronting said first connected end in a radial direction relative to the axis, and being attached to said first connected end at a bonding region with a bonding force such that the bonding force is greater than a frictional force generated between said deformable surrounding wall and said large-diameter wall portion when said plunger is forced to move in said accommodation chamber, and such that immediately after said second connected end stays put as a result of abutment of said head end wall against said shoulder, a subsequent forward force applied to said rear end wall of the plunger imparts to said bonding region a shear force which breaks said bonding region when the shear force is greater than the bonding force, thereby preventing operation of said plunger to move said deformable sealing member rearward, and thereby rendering said syringe unreusable.

2. The single-use syringe of claim 1, wherein said second connected end of said second coupling member has an outer sliding surface which extends in the axial direction so as to guide said bonding region to slide thereon once said bonding region is broken by the subsequent forward force.

3. The single-use syringe of claim 2, wherein said outer sliding surface has an inclined segment which is disposed proximate to said second secured end and which is inclined from the axis to said second secured end such that when said bonding region slides along said inclined segment, said second connected end is bent toward the axis so as to acquire a biasing force that imparts to said bonding region an increased force component in the axial direction to thereby facilitate rearward movement of said bonding region relative to said inclined segment.

4. The single-use syringe of claim 3, wherein said second coupling member has left and right bendable units which cooperatively serve as said second connected end, and which are spaced apart from each other in a radial direction to define an axial clearance therebetween so as to provide a leeway for said left and right bendable units to be bent toward the axis; and
wherein said first coupling member has left and right sliding units which cooperatively serve as said first connected end, and which confront respectively said left and right bendable units in the radial direction, and which are spaced apart from each other in the radial direction to confine therebetween an axial channel so as to accommodate said left and right bendable units when said bonding region slides along said inclined segment.

5. The single-use syringe of claim 1, further comprising a plug which is connected to one of said head end wall of said deformable sealing member and said second secured end of said second coupling member, and which extends forwardly from said head end wall such that when said head end wall is brought to abut against said shoulder, said plug is forced to plug said passage, said plug having frictional ribs that are configured to frictionally engage said small-diameter wall portion with enhanced friction.

6. The single-use syringe of claim 1, further comprising an encasing member which is configured to be sleevable on said surrounding barrel wall from said front barrel end, and which includes
a surrounding front segment that surrounds said small-diameter wall portion, and that permits said needle cannula to extend outwardly thereof and to be retractable thereinto, and
a surrounding rear segment that extends from said surrounding front segment in the longitudinal direction to be sleeved on said large-diameter wall portion, and that is movable relative to said barrel between a position of use, where said small-diameter wall portion is closer to said surrounding front segment, and where said needle cannula extends outwardly of said surrounding front segment, and a disposal position, where said small-diameter wall portion is remote from said surrounding front segment, and where said needle cannula is disposed inwardly of said surrounding front segment as a result of withdrawal of said small-diameter wall portion from said surrounding front segment.

7. The single-use syringe of claim 6, wherein said large-diameter wall portion and said rear surrounding segment respectively have a spirally extending recess and a protrusion which are angularly engageable when said encasing member is sleeved on said barrel so as to prevent said surrounding barrel wall from displacing along said surrounding rear segment in the axial direction, thereby placing said surrounding barrel wall in the position of use, and which are configured such that said surrounding barrel wall is angularly movable relative to said surrounding rear segment so as to enable subsequent axial displacement of said surrounding barrel wall from the position of use to the disposal position.

8. The single-use syringe of claim 7, wherein said large-diameter wall portion has a surrounding retaining groove which is formed adjacent to said small-diameter wall portion and which is opposite to said spirally extending recess in the axial direction so as to engage said protrusion when said surrounding barrel wall is in the disposal position, thereby preventing said surrounding barrel wall from displacing along said surrounding rear segment.

9. The single-use syringe of claim 8, wherein said large-diameter wall portion further has a guideway which extends to interconnect said spirally extending recess and said surrounding retaining groove so as to facilitate the displacement of said surrounding barrel wall from the position of use to the disposal position.

10. The single-use syringe of claim 5, further comprising a tip protector which is detachably sleeved on said surrounding front segment of said encasing member for shielding said needle cannula.

11. The single-use syringe of claim 1, wherein said surrounding barrel wall has a larger thickness at said small-diameter wall portion, and a smaller thickness at said large-diameter wall portion.

12. The single-use syringe of claim 11, wherein said small-diameter wall portion has an inner grip surface which defines said passage and which is configured such that said rear secured end of said needle cannula is in a fluid-tight engagement with said inner grip surface.

13. The single-use syringe of claim 11, further comprising a tip protector which is detachably sleeved on said small-diameter wall portion for shielding said needle cannula and which is disposed to be in a fluid-tight engagement with a portion of said large-diameter wall portion.

14. The single-use syringe of claim 5, wherein said passage includes a wider passage segment which extends from said shoulder forwardly and which is dimensioned such that said plug is tightly fitted thereinto, and a narrower passage segment which is disposed between said wider passage segment and said front barrel end.

15. The single-use syringe of claim 14, wherein said small-diameter wall portion has an inner grip surface which defines said narrower passage segment and which is configured such that said rear secured end of said needle cannula is in a fluid-tight engagement with said inner grip surface.

16. The single-use syringe of claim 1, further comprising:
a tubular needle seat having a tubular retaining portion which retains said rear secured end of said needle cannula to permit forward extension of said front tip end from said tubular retaining portion, and a tubular sleeve portion which is opposite to said tubular retaining portion along the axis to define a duct that is fluidly communicated with said needle cannula, and which is detachably sleeved on said small-diameter wall portion to fluidly communicate said duct with said passage; and
a tip protector which is removable and is in a fluid-tight engagement with said tubular sleeve portion for shielding said needle cannula.

17. The single-use syringe of claim 1, wherein said barrel includes a tubular guard portion which extends rearwardly from said rear barrel end and which defines a receiving space communicated with said accommodation chamber, said plunger having a thumb rest which extends rearwardly and outwardly of said tubular guard portion for manual operation, and which is received in said receiving space when said deformable sealing member reaches said shoulder.
